(19) United States
(12) Patent Application Publication
(10) Pub. No.: US 2009/0324534 A1
(43) Pub. Date: Dec. 31, 2009

(54) SOLID EMULSION CONTAINING A LIQUID FATTY PHASE STRUCTURED WITH A POLYMER

(75) Inventor: Patricia LEMANN, Creteil (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: L'Oreal S.A.

(21) Appl. No.: 12/543,346

(22) Filed: Aug. 18, 2009

Related U.S. Application Data
(63) Continuation of application No. 10/312,083, filed on Mar. 26, 2003, now abandoned.

(30) Foreign Application Priority Data
Jun. 23, 2000 (FR) 00/08084

(51) Int. Cl. A61K 8/72

(52) U.S. Cl. 424/78.03

(57) ABSTRACT

The invention relates to a physiologically acceptable solid emulsion, especially a cosmetic emulsion, containing an aqueous phase and a liquid fatty phase, one dispersed in the other, said liquid fatty phase being structured with at least one polymer having a weight-average molecular mass ranging from 1000 to 30 000, comprising a) a polymer skeleton containing hydrocarbon-based repeating units provided with at least one heteroatom, and b) optionally functionalized pendent and/or terminal fatty chains containing from 12 to 120 carbon atoms and being linked to these units, the aqueous phase, the liquid fatty phase and the polymer forming a physiologically acceptable medium. This composition is especially in the form of a cast foundation, the application of which produces a natural, light, fresh deposit that shows good staying power over time.

The present invention relates to a care and/or treatment and/or makeup composition for the skin, including the scalp, and/or for the lips of human beings, of emulsion type, containing a liquid fatty phase structured with a particular polymer. This composition is especially in the form of a makeup product in particular cast in stick or dish form, and more especially a concealer stick, a body makeup, a foundation or a lip product, the application of which produces a natural, light deposit that has good staying power over time. More especially, this composition is in the form of a water-in-oil (W/O) solid emulsion.

It is common to find a liquid fatty phase that is structured, i.e. gelled and/or rigidified with the aid of waxes and/or fillers, in solid cosmetic or dermatological products; this is especially the case in solid compositions such as deodorants, lip balms, lipsticks, concealer products, eyeshadows and foundations cast in a jar or dish. Products cast in a dish are often referred to as compacts.

For the purposes of the invention, the expression “liquid fatty phase” means a fatty phase that is liquid at room temperature (25° C.) and atmospheric pressure (760 mmHg), composed of one or more nonaqueous compounds that are liquid at room temperature, also referred to as oils, which are generally mutually compatible and at least partially insoluble in an aqueous phase. The nonaqueous compounds or oils may be volatile or nonvolatile. Thus, the liquid fatty phase may contain one or more volatile compounds, one or more nonvolatile compounds or a mixture of these volatile and nonvolatile compounds.

For the purposes of the invention, the term “volatile compound” means any nonaqueous medium capable of evaporating on contact with the skin or the lips in less than one hour, at room temperature and atmospheric pressure. The volatile solvent(s) of the invention is (are) organic solvents and especially volatile cosmetic oils, that are liquid at room temperature, having a nonzero vapor pressure, at room temperature and atmospheric pressure, ranging in particular from 10⁻³ to 300 mmHg and preferably greater than 0.3 mmHg.

The structuring of the liquid fatty phase makes it possible in particular to limit its exudation from solid compositions, in particular in hot and humid regions, and in addition to limit, after deposition on the skin or the lips, the migration or propagation of this phase in the wrinkles and fine lines, which is particularly desired for a lipstick, a concealer product or an eyeshadow. The term “migration” means a running of the liquid fatty phase beyond the initial application line of the makeup. This migration of the liquid fatty phase, in particular when it is large and when this phase is charged with dyestuffs, leads to an unattractive effect around the lips and the eyes, which particularly accentuates the wrinkles and fine lines. This migration is often mentioned by women as being a major defect of colored makeup products, for instance standard lipsticks, concealer products and eyeshadows.

Furthermore, cast care or makeup compositions are usually in anhydrous form, resulting, in particular on the face and the body, but also on the eyelashes, in phenomena of discomfort, heaviness, greasiness and occasionally sensations of wearing a mask or of stifling, which may become unacceptable.

To lighten the makeup and reduce these phenomena of heaviness and greasiness, cosmeticians directed their attention toward foundation and lipstick compositions in emulsion form. Thus, the company Shiseido envisaged, in its patent application EP-A-0 374 332, lipstick and foundation compositions that produce a fresh effect, in the form of a solid emulsion of the water-in-oil type containing a volatile silicone oil, a solid hydrocarbon-based wax, an organopolysiloxane modified with a polyoxyalkylene group, serving as an emulsifier for the aqueous phase in the fatty phase, and pulverulent fillers. Similarly, the company Procter & Gamble described, in document U.S. Pat. No. 5,688,831, moisturizing makeup compositions comprising one or more volatile silicones combined with one or more humectants, pigments and an organic amphiphilic compound capable of forming on the skin or in the composition smectic lyotropic liquid crystals containing said humectants.

Due to the presence of a high content of wax, these compositions still have the drawback of being heavy, of being difficult to spread and of usually giving the composition an unpleasant feel. Furthermore, the presence of these waxes generally causes matting of the makeup, which is not always desirable for a lipstick or an eyeshadow.

There is thus still the need for a composition that does not have the above drawbacks, that especially has properties of freshness, lightness and good staying power over time, and that does not dry out the skin or the lips onto which it is applied, either during the application or over time. Furthermore, this composition should be stable over time and easy to manufacture. The makeup result obtained should be natural, should not have the sensation of a mask or of stifling, and should be uniform.

One subject of the invention is, precisely, a solid emulsion for caring for and/or making up and/or treating the skin and/or the lips of the face and/or the integuments, which makes it possible to overcome the drawbacks mentioned above.

The term “emulsion” means a composition containing an aqueous phase and a liquid fatty phase, one of the two phases of which is dispersed in the other phase with or without an emulsifier, the mixture being homogeneous to the naked eye. The term “solid emulsion” means an emulsion that does not flow under its own weight at room temperature and atmospheric pressure.

The Applicant has found, surprisingly, that the use of particular polymers in a solid emulsion containing an aqueous phase and a fatty phase makes it possible to obtain a cast or compact product whose application to the skin, the lips or keratin fibers leads to a film that has noteworthy cosmetic properties. In particular, the film is supple, comfortable and “fresh”. Furthermore, the composition (or the emulsion) is stable over time and does not exude at room temperature.

The term “stable” refers to a composition that does not exude at room temperature for at least two months, or even up to nine months.

The invention applies not only to lip makeup products, for instance lipsticks, lip glosses and lip pencils and to skin makeup products, for either the human face or body, for instance foundations optionally cast as sticks, in jars or in dishes, concealer products, eyeshadows, rouges and transfer tattoo products, but also to care and/or treatment products for the skin, including the scalp, and for the lips, for instance antisun products especially in stick form for protecting facial skin or the lips, body hygiene products, for instance deodorants, especially in stick form, thickened shampoos and conditioners, and eye makeup products, for instance eyeliners, pencils and mascaras more especially cast in cake form, and also makeup or care products for keratin fibers such as the hair and the eyebrows.

More specifically, one subject of the invention is a solid emulsion containing an aqueous phase and a liquid fatty phase, one dispersed in the other, the liquid fatty phase being structured with at least one polymer having a weight-average molecular mass of less than 100 000 and especially ranging from 1000 to 30 000, comprising a) a polymer skeleton containing hydrocarbon-based repeating units provided with at least one heteroatom, and b) optionally functionalized pendent and/or terminal fatty chains containing from 12 to 120 carbon atoms and being linked to these hydrocarbon-based units, the aqueous phase, the liquid fatty phase and the polymer forming a physiologically acceptable medium.

More especially, the weight-average molecular mass is less than 100 000 and better still less than 50 000.

The composition of the invention may be in the form of an optionally deformable, more of less hard paste or solid. It may be an oil-in-water (O/W) or water-in-oil (W/O) emulsion, or even in the form of a triple emulsion. In particular, it is in cast form as a stick or in a dish and more especially in the form of a rigid water-in-oil emulsion, especially a cast emulsion.

The structuring of the liquid fatty phase may be modified depending on the nature of the heteroatom-bearing polymer used and may be such that a more or less hard structure is obtained, especially in the form of a tube or a stick. When these rigid compositions are colored, they make it possible, after application, to obtain a deposit that is uniform in color, more or less glossy, fresh, not heavy, and supple, which does not migrate and which shows good resistance, especially of the color over time.

The structuring polymer in the composition of the invention is a solid that is undeformable at room temperature (25° C.) and atmospheric pressure (760 mmHg, i.e. about 1.013×10⁵ Pa). It is capable of structuring the composition without opacifying it.

For the purposes of the invention, the expression “functionalized chains” means an alkyl chain comprising one or more functional or reactive groups chosen in particular from hydroxyl, ether, oxyalkylene, polyoxyalkylene, halogen, including fluoro or perfluoro, ester, siloxane and polysiloxane groups. In addition, the hydrogen atoms of one or more fatty chains may be substituted at least partially with fluorine atoms.

For the purposes of the invention, the term “polymer” means a compound containing at least two repeating units.

According to the invention, the expression “hydrocarbon-based repeating units” means a unit containing from 2 to 80 carbon atoms and preferably from 2 to 60 carbon atoms, bearing hydrogen atoms and possibly oxygen atoms, which may be linear, branched or cyclic, and saturated or unsaturated. These units each also comprise one or more heteroatoms that are non-pendent and are in the polymer skeleton. These heteroatoms are chosen from nitrogen, sulfur and phosphorus atoms and combinations thereof, optionally combined with one or more oxygen atoms. These units may also comprise a polar group of carbonyl type.

These units containing a heteroatom are, in particular, amide units forming a skeleton of the polyamide type, carbamate and/or urea units forming a polyurethane, polyurea and/or polyurea-urethane skeleton. These units are preferably amide units. The pendent chains are advantageously linked directly to at least one of the heteroatoms of the polymer skeleton.

Between the hydrocarbon-based units, the polymer may comprise silicone units or oxyalkylene units.

In addition, the polymer in the composition of the invention advantageously comprises from 40% to 98% of fatty chains relative to the total number of units containing a heteroatom and of fatty chains, and better still from 50% to 95%. The nature and proportion of the units containing a heteroatom depend on the nature of the liquid fatty phase and is, in particular, similar to the nature of the fatty phase. Thus, the more the units containing a heteroatom are polar and in high proportion in the polymer, which corresponds to the presence of several heteroatoms, the greater the affinity of the polymer for polar oils. Conversely, the more nonpolar, or even apolar, the units containing a heteroatom, or the lower the proportion thereof, the greater the affinity of the polymer for apolar oils.

A subject of the invention is also a solid emulsion containing an aqueous phase and a liquid fatty phase, one dispersed in the other, the liquid fatty phase being structured with at least one polyamide having a weight-average molecular mass of less than 100 000 and especially ranging from 1000 to 30 000, comprising a) a polymer skeleton containing amide repeating units, and b) possibly optionally functionalized pendent and/or terminal fatty chains containing from 8 to 120 carbon atoms and being linked to these amide units, the aqueous phase, the liquid fatty phase and the polymer forming a physiologically acceptable medium.

This emulsion may be used in unmodified form or introduced into a more complex composition containing a physiologically acceptable medium.

The pendent fatty chains are preferably linked to at least one of the nitrogen atoms of the amide units.

In particular, the fatty chains of this polyamide represent from 40% to 98% of the total number of amide units and of fatty chains, and better still from 50% to 95%.

Advantageously, the first polymer, and in particular the polyamide, of the composition according to the invention has a weight-average molecular mass of less than 100 000 (especially ranging from 1000 and 100 000), in particular less than 50 000 (especially ranging from 1000 to 50 000) and more particularly ranging from 1000 to 30 000, preferably from 2000 to 20 000 and better still from 2000 to 10 000.

As preferred structuring polymers that may be used in the invention, mention may be made of polyamides branched with pendent fatty chains and/or terminal fatty chains containing from 12 to 120 carbon atoms and especially from 12 to 68 carbon atoms, the terminal fatty chains being linked to the polyamide skeleton via ester groups.

These first polymers are preferably polymers resulting from a polycondensation between a dicarboxylic acid containing at least 32 carbon atoms (in particular containing from 32 to 44 carbon atoms) and a diamine containing at least 2 carbon atoms (in particular from 2 to 36 carbon atoms). The diacid is preferably a dimer derived from an ethylenically unsaturated fatty acid containing at least 16 carbon atoms and preferably from 16 to 24 carbon atoms, for instance oleic acid, linoleic acid or linolenic acid. The diamine is preferably ethylenediamine, hexylenediamine or hexamethylenediamine. For the polymers comprising one or 2 terminal carboxylic acid groups, it is advantageous to esterify them with a monoalcohol containing at least 4 carbon atoms, preferably from 10 to 36 carbon atoms, better still from 12 to 24 and even better from 16 to 24, for example 18 carbon atoms.

These polymers are more especially those disclosed in document U.S. Pat. No. 5,783,657 from the company Union Camp. Each of these polymers in particular satisfies formula (I) below:

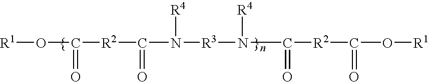

in which n denotes a whole number of amide units such that the number of ester groups represents from 10% to 50% of the total number of ester and amide groups; R₁ is, independently in each case, an alkyl or alkenyl group containing at least 4 carbon atoms and in particular from 4 to 24 carbon atoms; R² represents, independently in each case, a saturated or unsaturated C₄ to C₄₂ hydrocarbon-based group, on condition that 50% of the groups R² represent a saturated or unsaturated C₃₀ to C₄₂ hydrocarbon-based group; R³ represents, independently in each case, an organic group containing at least 2 carbon atoms, hydrogen atoms and optionally one or more oxygen or nitrogen atoms; and R⁴ represents, independently in each case, a hydrogen atom, a saturated or unsaturated C₁ to C₁₀ alkyl group or a direct bond to R³ or to another R⁴, such that the nitrogen atom to which R³ and R⁴ are both attached forms part of a heterocyclic structure defined by R⁴—N—R³, with at least 50% of the groups R⁴ representing a hydrogen atom.

In the particular case of formula (I), the terminal fatty chains that are optionally functionalized for the purposes of the invention are terminal chains linked to the last heteroatom, in this case nitrogen, of the polyamide skeleton.

In particular, the ester groups of formula (I), which form part of the terminal and/or pendent fatty chains for the purposes of the invention, represent from 15% to 40% of the total number of ester and amide groups and better still from 20% to 35%. Furthermore, n advantageously represents an integer ranging from 1 to 5. Preferably, R¹ is a saturated or unsaturated C₁₂ to C₂₂ and preferably C₁₆ to C₂₂ alkyl group. Advantageously, R² can be a C₁₀ to C₄₂ hydrocarbon-based (alkylene) group. Preferably, at least 50% and better still at least 75% of the groups R² are groups containing from 30 to 42 carbon atoms. The other groups R² are C₄ to C₁₉ and better still C₄ to C₁₂ hydrogen-containing groups. Preferably, R³ represents a C₂ to C₃₆ hydrocarbon-based group or a polyoxyalkylene group and R⁴ represents a hydrogen atom. Preferably, R³ represents a saturated or unsaturated C₂ to C₁₂ hydrocarbon-based group. The hydrocarbon-based groups may be linear, cyclic or branched, and saturated or unsaturated groups. Moreover, the alkyl and alkylene groups may be linear or branched groups.

Advantageously, the polymer in the composition of the invention has a weight-average molecular mass ranging from 2000 to 20 000 and better still from 2000 to 10 000.

According to the invention, the structuring of the liquid fatty phase is obtained with the aid of one or more polymers of formula (I). In general, the polymers of formula (I) are in the form of mixtures of polymers, these mixtures also possibly containing a synthetic product of formula (I) corresponding to a compound in which n is 0, i.e. a diester.

As examples of structuring polymers which can be used in the composition according to the invention, mention may be made of the commercial products manufactured and/or sold by the company Bush Boake Allen under the names Uniclear 80 and Uniclear 100. They are sold, respectively, in the form of an 80% (in terms of active material) gel in a mineral oil and a 100% (in terms of active material) gel. They have a softening point of from 88 to 94° C. These commercial products are a mixture of copolymers of a C₃₆ diacid coupled with ethylenediamine, having a weight-average molecular mass of about 6000. The terminal ester groups result from the esterification of the remaining acid endings with cetyl alcohol, stearyl alcohol or mixtures thereof (also known as cetylstearyl alcohol).

As structuring polymers which can be used in the invention, mention may also be made of polyamide resins resulting from the condensation of an aliphatic dicarboxylic acid and a diamine (including compounds containing, respectively, more than 2 carboxyl groups and more than 2 amine groups), the carboxyl and amine groups of adjacent individual units being condensed in the form of an amide bond. These polyamide resins are, in particular, those sold under the brand name Versamid® by the companies General Mills, Inc. and Henkel Corp. (Versamid 930, 744 or 1655) or by the company Olin Mathieson Chemical Corp. under the brand name Onamid®, in particular Onamid S or C. These resins have a weight-average molecular mass ranging from 6000 to 9000. For further information regarding these polyamides, reference may be made to the documents U.S. Pat. No. 3,645,705 and U.S. Pat. No. 3,148,125. More especially, Versamid® 930 or 744 is used.

The polyamides sold by the company Union Camp Corp. under the references Uni-Rez (2658, 2931, 2970, 2621, 2613, 2624, 2665, 1554, 2623 and 2662) and the product sold under the reference Macromelt 6212 by the company Henkel may also be used. For further information regarding these polyamides, reference may be made to document U.S. Pat. No. 5,500,209.

The structuring polymers in the composition of the invention advantageously have a softening point of greater than 65° C., which may be up to 190° C. They preferably have a softening point ranging from 70 to 130° C. and better still from 80 to 105° C. This softening point is lower than those of the known structuring polymers, thus facilitating the use of the polymers that are the subject of the invention and limiting the degradation of the liquid fatty phase. These polymers are, in particular, nonwaxy polymers.

The polymer is advantageously combined with at least one amphiphilic compound that is liquid and nonvolatile at room temperature, having a hydrophilic/lipophilic balance (HLB) of less than 12 and in particular ranging from 1 to 8 and preferably from 1 to 5. According to the invention, one or more amphiphilic compounds may be used. The aim of these amphiphilic compounds is to reinforce the structuring properties of the polymer containing a heteroatom, to facilitate the use of the polymer and to improve the ability to deposit of the solid emulsion.

According to the invention, the composition of emulsion type may have a hardness ranging from 10 g to 200 g and better still from 30 g to 100 g. This hardness may be measured according to a method of penetrating a probe into said composition and in particular using a texture analyzer (for example TA-XT2 from Rheo) equipped with an ebonite cylinder 5 mm tall and 8 mm in diameter. The hardness measurement is performed at 20° C. at the center of 5 samples of said composition. The cylinder is introduced into each sample of composition at a prespeed of 2 mm/s and then at a speed of 0.5 mm/s, and finally at a post-speed of 2 mm/s, the total displacement being 1 mm. The hardness value recorded is that of the maximum peak. The sample is about 1 cm thick.

The hardness of the composition (or the emulsion) according to the invention is such that the composition is self-supporting and can disintegrate easily to form a satisfactory deposit on the skin and/or the lips and/or the integuments. In addition, with this hardness, the composition of the invention has good impact strength.

According to the invention, the solid emulsion cast in particular as a dish or optionally in stick form has the behavior of a deformable, flexible elastic solid, giving noteworthy elastic softness on application. The cast compositions of the prior art do not have this property of elasticity and flexibility.

The amphiphilic compound(s) which can be used in the composition of the invention comprise a lipophilic part linked to a polar part, the lipophilic part comprising a carbon-based chain containing at least 8 carbon atoms, in particular from 18 to 32 carbon atoms and better still from 18 to 28 carbon atoms. The polar part of this or these amphiphilic compound(s) is preferably the residue of a compound chosen from alcohols and polyols containing from 1 to 12 hydroxyl groups, and polyoxyalkylenes comprising at least 2 oxyalkylene units and containing from 0 to 20 oxypropylene units and/or from 0 to 20 oxyethylene units. In particular, the amphiphilic compound is an ester chosen from the hydroxystearates, oleates and isostearates of glycerol, of sorbitan or of methylglucose, or alternatively branched C₁₂ to C₂₆ fatty alcohols such as octyldodecanol, and mixtures thereof. Among these esters, monoesters and mixtures of mono- and diesters are preferred.

The amount of amphiphilic compound and that of the heteroatom-bearing polymer are chosen according to the desired hardness of the gel and as a function of the specific application envisaged. The respective amounts of polymer and of amphiphilic compound should be such that they produce a stick that can be worn down. In practice, the amount of polymer (as active material) represents from 0.5% to 80% of the total weight of the composition and better still front 5% to 40%, or even from 7% to 20%. The amount of amphiphilic compound in practice represents from 0.1% to 35% of the total weight of the composition and better still from 1% to 15%, if it is present.

The liquid fatty phase of the composition advantageously contains at least 40% of liquid oil(s) containing a group similar to that of the units containing a heteroatom, and for example front 50% to 100%. In particular, the liquid fatty phase structured with a polyamide-type skeleton contains a high quantity, i.e. at least 40% of the total weight of the liquid fatty phase and better still from 50% to 100%, of liquid apolar and more especially hydrocarbon-based oil or mixture of oils.

For a liquid fatty phase structured with a polymer containing a partially silicone-based skeleton, this fatty phase preferably contains at least 40% relative to the total weight of the liquid fatty phase, and better still from 50% to 100%, of silicone-based liquid oil or mixture of oils, relative to the total weight of the liquid fatty phase.

For a liquid fatty phase structured with an apolar polymer of the hydrocarbon-based type, this fatty phase advantageously contains at least 40% by weight, and better still from 50% to 100% by weight, of liquid apolar and in particular hydrocarbon-based oil or mixture of oils, relative to the total weight of the liquid fatty phase.

The term “hydrocarbon-based oil” means any oil containing carbon and hydrogen atoms and possibly an ether, ester, acid or hydroxyl function.

In particular, the polar oils of the invention are:

hydrocarbon-based plant oils with a high content of triglycerides consisting of fatty acid esters of glycerol in which the fatty acids may have varied chain lengths from C₄ to C₂₄, these chains possibly being linear or branched, and saturated or unsaturated; these oils are, in particular, wheat germ oil, corn oil, sunflower oil, karite butter, castor oil, sweet almond oil, macadamia oil, apricot oil, soybean oil, cotton oil, alfalfa oil, poppy oil, pumpkin oil, sesame oil, marrow oil, rapeseed oil, avocado oil, hazelnut oil, grape seed oil, blackcurrant seed oil, evening primrose oil, millet oil, barley oil, quinoa oil, olive oil, rye oil, safflower oil, candlenut oil, passion flower oil and musk rose oil; or alternatively caprylic/capric acid triglycerides such as those sold by the company Stearineries Dubois or those sold under the names Miglyol 810, 812 and 818 by the company Dynamit Nobel;

synthetic oils or esters of formula R₁COOR₂ in which R₁ represents a linear or branched fatty acid residue containing from 1 to 40 carbon atoms and R₂ represents an in particular branched hydrocarbon-based chain containing from 1 to 40 carbon atoms, with R₁+R₂≧10, such as, for example, purcellin oil (cetostearyl octanoate), isononyl isononanoate, C₁₂-C₁₅ alkyl benzoate, isopropyl myristate, 2-ethylhexyl palmitate, isostearyl isostearate, and alkyl or polyalkyl octanoates, decanoates or ricinoleates; hydroxylated esters such as isostearyl lactate and diisostearyl malate; and pentaerythritol esters;

synthetic ethers containing from 10 to 40 carbon atoms;

C₈ to C₂₆ fatty alcohols such as oleyl alcohol;

mixtures thereof.

The apolar oils according to the invention are, in particular, silicone oils such as volatile or nonvolatile, linear or cyclic polydimethylsiloxanes (PDMSs) that are liquid at room temperature; polydimethylsiloxanes comprising alkyl, alkoxy or phenyl groups which are pendent and/or at the end of the silicone chain, the groups each containing from 2 to 24 carbon atoms; phenylsilicones such as phenyltrimethicones, phenyldimethicones, phenyltrimethylsiloxydiphenylsiloxanes, diphenyldimethicones, diphenylmethyldiphenyltrisiloxanes and 2-phenylethyl trimethylsiloxysilicates; linear or branched, volatile or nonvolatile hydrocarbons of synthetic or mineral origin, such as volatile or nonvolatile liquid paraffins and derivatives thereof, petroleum jelly, liquid lanolin, polydecenes, hydrogenated polyisobutene such as parleam, and squalane, and mixtures thereof. The structured oils, and more especially those structured with polyamides and in particular those of formula (I) or the polyurethanes or polyureas or polyurea-polyurethanes, are preferably apolar oils and more especially an oil or a mixture of oils of the hydrocarbon-based type of mineral or synthetic origin, chosen in particular from hydrocarbons, especially alkanes such as parleam oil, isoparaffins such as isododecane, and squalane, and mixtures thereof. These oils are advantageously combined with one or more phenylsilicone oils.

The liquid fatty phase preferably contains at least one nonvolatile oil chosen in particular from hydrocarbon-based oils of mineral, plant or synthetic origin, synthetic esters or ethers and silicone oils, and mixtures thereof.

In practice, the total liquid fatty phase represents from 5% to 98% and preferably from 20% to 75% relative to the total weight of the composition.

The liquid fatty phase in the composition according to the invention may also contain at least one volatile nonaqueous compound, i.e. one or more volatile compounds. The volatile compounds are generally solvents for the nonvolatile compounds.

According to the invention, these volatile compounds facilitate, in particular, the application of the composition to the skin, the lips or integuments. These volatile compounds may be hydrocarbon-based compounds, silicone compounds optionally comprising alkyl or alkoxy groups that are pendent or at the end of a silicone chain, or a mixture of these volatile compounds.

As volatile compounds that may be used in the invention, mention may be made of linear or cyclic silicone oils having a viscosity at room temperature of less than 10 cSt and in particular containing from 2 to 7 silicon atoms, these silicones optionally comprising alkyl or alkoxy groups containing from 1 to 10 carbon atoms. As volatile silicone oils which may be used in the invention, mention may be made in particular of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

As other volatile compounds that may be used in the invention, mention may be made of hydrocarbon-based volatile oils containing from 8 to 16 carbon atoms, and mixtures thereof, and in particular branched C₈-C₁₆ alkanes, for instance C₈-C₁₆ isoalkanes (also known as isoparaffins), isododecane, isodecane, isohexadecane and, for example, the oils sold under the trade names “Isopars” or “Permethyls”, and branched C₈-C₁₆ esters, for instance isohexyl neopentanoate, and mixtures thereof. The volatile compound (or solvent) is preferably chosen from hydrocarbon-based volatile oils containing from 8 to 16 carbon atoms, and mixtures thereof.

Volatile fluoro compounds may also be used.

These volatile compounds in particular represent a mass content of from 0% to 97.5% relative to the total weight of the composition, preferably from 1% to 75% and better still from 3% to 45% (if present). In general, the amount of volatile compound used is an amount that is sufficient to obtain sliding and spreading properties. This amount will be adapted by a person skilled in the art according to the desired intensity of the spreading properties.

The composition of the invention also contains an aqueous phase that is immiscible with the liquid fatty phase, containing water optionally thickened or gelled with one or more aqueous-phase thickeners or gelling agents and optionally containing water-miscible compounds, for instance C₂ to C₇ lower alcohols, polyols containing at least two hydroxyl groups and from 2 to 10 carbon atoms, for instance glycerol, propylene glycol, D-panthenol or polyethylene glycols. The aqueous phase represents especially from 1% to 80% and better still from 5% to 70% of the total weight of the composition.

The emulsions according to the invention may be obtained using a surfactant or a mixture of surfactants whose HLB (hydrophilic/lipophilic balance) value is adapted to the sense of the emulsion.

As surfactants that may be used in the invention, suitable for obtaining a W/O emulsion, mention may be made of those with an HLB value of less than 7, and especially fatty acid esters of polyols, for instance mono-, di-, tri- or sesquioleates or stearates of sorbitol or of glycerol, glyceryl laurate or polyethylene glycol laurate; alkyl or alkoxy dimethicone copolyols with an alkyl or alkoxy chain that is pendent or at the end of the silicone skeleton, containing, for example, from 6 to 22 carbon atoms. As surfactants that may be used to obtain an O/W emulsion, mention may be made of those with an HLB value of greater than 7, for instance fatty acid esters of polyethylene glycol (polyethylene glycol monostearate or monolaurate); polyoxyethylenated fatty acid esters (stearate or oleate) of sorbitol; polyoxyethylenated alkyl (lauryl, cetyl, stearyl or octyl)ethers and dimethicone copolyols. In general, any amphoteric ionic (cationic or anionic) surfactant and any nonionic surfactant that is well known to those skilled in the art may be used.

The composition of the invention may also comprise any additive usually used in the field under consideration, chosen in particular from dyestuffs, antioxidants, essential oils, preserving agents, fragrances, electrolytes (cations or polycations), fillers, waxes, products that are pasty at room temperature, neutralizing agents, liposoluble polymers or polymers that are dispersible in the medium, cosmetic or dermatological active agents such as, for example, emollients, moisturizers, vitamins, essential fatty acids, sunscreens, dispersants such as poly(12-hydroxystearic acid), and mixtures thereof. These additives may be present in the composition in a proportion of from 0% to 20% (in particular from 0.01% to 20%) relative to the total weight of the composition and better still from 0.01% to 10%. The composition advantageously contains at least one cosmetic or dermatological active agent.

Needless to say, a person skilled in the art will take care to select the optional additional additives and/or the amount thereof such that the advantageous properties of the composition according to the invention are not, or are not substantially, adversely affected by the envisaged addition.

The composition according to the invention can be in the form of a tinted dermatological composition or a care composition for keratin materials such as the skin, the lips and/or the integuments, such as keratin fibers, in the form of an antisun composition or body hygiene composition in particular in the form of a deodorant product or makeup remover in stick form. It can be used in particular as a care base for the skin, the integuments or the lips (lip balms, for protecting the lips against cold and/or sunlight and/or the wind, or a care product for the skin, the nails or the hair).

The composition of the invention may also be in the form of a colored makeup product for the skin, in particular a foundation, optionally having care or treating properties, a blusher, a face powder, an eyeshadow, a concealer product, an eyeliner, a makeup product for the body; a makeup product for the lips such as a lipstick, a lip gloss or a lip pencil optionally having care or treating properties; a makeup product for integuments such as the eyelashes and the hair, in particular in the form of a mascara cake, for the eyebrows, in particular in the form of a pencil, or for the nails.

Needless to say, the composition of the invention should be cosmetically or dermatologically acceptable, i.e. it should contain a nontoxic physiologically acceptable medium and should be able to be applied to the skin, the integuments or the lips of human beings. For the purposes of the invention, the expression “cosmetically acceptable” means a composition of pleasant appearance, odor and feel.

The composition advantageously contains at least one cosmetic active agent and/or one dermatological active agent and/or at least one dyestuff. By means of the use of at least one polymer with a weight-average molecular mass ranging from 1000 to 30 000, as defined above, in an emulsion, trapping of the active agents and dyestuffs present in the composition is obtained, allowing them to be kept in the place that they were applied, i.e. the lips, the skin or the integuments.

The dyestuff according to the invention may be chosen from the lipophilic dyes, hydrophilic dyes, pigments and nacres usually used in cosmetic or dermatological compositions, and mixtures thereof. This dyestuff is generally present in a proportion of from 0.01% to 50% relative to the total weight of the composition, preferably from 0.5% to 30%, if it is present.

The liposoluble dyes are, for example, Sudan Red, D&C Red 17, D&C Green 6, β-carotene, soybean oil, Sudan Brown, D&C Yellow 11, D&C Violet 2, D&C Orange 5 or quinoline yellow. They can represent from 0.1% to 20% of the weight of the composition and better still from 0.1% to 6% (if present).

The pigments may be white or colored, mineral and/or organic, and coated or uncoated. Among the mineral pigments which may be mentioned are titanium dioxide, optionally surface-treated, zirconium oxide or cerium oxide, as well as iron oxide, chromium oxide, manganese violet, ultramarine blue, chromium hydrate and ferric blue. Among the organic pigments that may be mentioned are carbon black, pigments of D & C type, and lakes based on cochineal carmine or on barium, strontium, calcium or aluminum. The pigments can represent from 0.1% to 50%, and better still from 2% to 30% relative to the total weight of the composition, if they are present.

The nacreous pigments may be chosen from white nacreous pigments such as mica coated with titanium or with bismuth oxychloride, colored nacreous pigments such as titanium mica with iron oxides, titanium mica with, in particular, ferric blue or chromium oxide, titanium mica with an organic pigment of the type mentioned above, as well as nacreous pigments based on bismuth oxychloride. They can represent from 0.1% to 20% relative to the total weight of the composition, and better still from 0.1% to 15%, if they are present.

The composition may optionally contain one or more waxes to improve the structuring in stick form, although this rigid form can be obtained in the absence of wax. For the purposes of the present invention, a wax is a lipophilic fatty compound that is solid at room temperature (25° C.), which undergoes a reversible solid/liquid change of state, having a melting point of greater than 40° C. and which may be up to 200° C., and having an anisotropic crystal organization in the solid state. The size of the crystals is such that the crystals diffract and/or scatter light, giving the composition a cloudy, more or less opaque appearance. By bringing the wax to its melting point, it is possible to make it miscible with oils and to form a microscopically homogeneous mixture, but on returning the temperature of the mixture to room temperature, recrystallization of the wax in the oils of the mixture is obtained. It is this recrystallization in the mixture that is responsible for the reduction in the gloss of said mixture. Thus, the composition advantageously contains little or no wax.

For the purposes of the invention, the waxes are those generally used in cosmetics and dermatology; they are especially of natural origin, for instance beeswax, carnauba wax, candelilla wax, ouricury wax, Japan wax, cork fiber wax, sugar cane wax, paraffin wax, lignite wax, microcrystalline waxes, lanolin wax, montan wax, ozokerites and hydrogenated oils such as hydrogenated jojoba oil as well as waxes of synthetic origin, for instance polyethylene waxes derived from the polymerization of ethylene, waxes obtained by Fischer-Tropsch synthesis, fatty acid esters and glycerides that are solid at 40° C., silicone waxes such as alkyl-, alkoxy- or esters of poly(di)methylsiloxane which is solid at 40° C.

The composition according to the invention may be manufactured by the known processes that are generally used in cosmetics or dermatology. It may be manufactured by the process that consists in heating the polymer at least to its softening point, in adding the amphiphilic compound(s), the emulsifiers, the dyestuffs and the additives thereto optionally in the molten state and then in mixing everything together until a clear, transparent solution is obtained. After reducing the temperature, the volatile compound(s), and then the aqueous phase, are then added to the mixture obtained. The mixture is then homogenized. The homogeneous mixture obtained can then be cast in a suitable mould such as a dish or a lipstick mould or directly into the packaging articles (case or dish in particular).

A subject of the invention is also a care, makeup or treatment cosmetic process for human keratin materials, and in particular the skin, the lips and the integuments, comprising the application to the keratin materials of the composition, in particular the cosmetic composition, as defined above.

The invention is illustrated in greater detail in the examples that follow. The percentages are given on a weight basis.

### EXAMPLE 1

### W/O Foundation

Phase A

| | |
|---|---|
| Sorbitan isostearate (Arlacel 987 from Uniqema) | 4.5% |
| Preserving agent | 0.2% |

Phase B

| | |
|---|---|
| Uniclear 80 | 15% |
| 2-Octyldodecanol | 3.6% |
| Hydrogenated isoparaffin (parleam) | 11% |

Phase C

| | |
|---|---|
| Titanium dioxide | 4.1% |
| Hydrogenated isoparaffin | 7% |
| Iron oxide | 1.3% |

Phase D

| | |
|---|---|
| Fragrance | 0.65% |

Phase E

| | |
|---|---|
| Water | qs 100% |
| Preserving agent | 0.3% |
| Magnesium sulfate | 0.7% |
| Propylene glycol | 3% |

Phase (B) is heated to 120° C., with stirring, and then homogenized using a Raineri homogenizer rotating at 300 rpm. Phase A is then added, followed by phase C which has been preground using a three-roll mill. Phase D and phase E are then added carefully and uniformly into the above mixture brought to 100° C. with a Heidolph homogenizer. The mixture is left in contact for 10 minutes and then poured into dishes.

The product obtained is solid and easy to take up with the fingers; it is fresh and spreads well, and forms a light, uniform deposit on the skin. The makeup result obtained is natural and satiny. Its hardness in g (using a texturometer) is 35.

### EXAMPLE 2

### Solid W/O Foundation

Phase A

| | |
|---|---|
| Sorbitan isostearate (Arlacel 987 from Uniqema) | 4.5% |
| Preserving agent | 0.2% |

Phase B

| | |
|---|---|
| Uniclear 80 | 15% |
| 2-Octyldodecanol | 3.6% |
| Hydrogenated isoparaffin (parleam) | 6% |
| Isododecane | 5% |

Phase C

| | |
|---|---|
| Titanium dioxide | 4.1% |
| Hydrogenated isoparaffin | 7% |
| Iron oxide | 1.3% |
| Nylon powder | 8% |

Phase D

| | |
|---|---|
| Fragrance | 0.65% |

Phase E

| | |
|---|---|
| Water | qs 100% |
| Preserving agent | 0.3% |
| Magnesium sulfate | 0.7% |
| Propylene glycol | 3% |

The manufacture of this foundation is identical to that of example 1. The product obtained has attractive solid texture and has the same properties as the product of example 1.

### EXAMPLE 3

### W/O Foundation

Phase A

| | |
|---|---|
| Sorbitan isostearate (Arlacel 987 from Uniqema) | 6% |
| Preserving agent | 0.2% |

Phase B

| | |
|---|---|
| Uniclear 80 | 15% |
| 2-Octyldodecanol | 3.6% |
| Hydrogenated isoparaffin (parleam) | 11% |
| Isododecane | 22.5% |
| Cyclohexadimethylsiloxane (8 cSt) | 5% |
| (DC 246 from Dow Corning) | |

Phase C

| | |
|---|---|
| Titanium dioxide | 4.1% |
| Hydrogenated isoparaffin | 3.3% |
| Iron oxide | 10.7% |
| Nylon powder | 4% |

Phase D

| | |
|---|---|
| Fragrance | 0.65% |

Phase E

| | |
|---|---|
| Water | qs 100% |
| Preserving agent | 0.2% |
| Magnesium sulfate | 0.7% |

The product obtained again has an attractive solid texture and has the sane cosmetic properties as the product of example 1.

### EXAMPLE 4

### W/O Foundation

This foundation differs from that of example 1 by the use of 17% Uniclear 80 instead of 15%. The product obtained is solid and has a hardness in g of 49.2.

1. A solid emulsion containing an aqueous phase and a liquid fatty phase, one dispersed in the other, the liquid fatty phase being structured with at least one polymer having a weight-average molecular mass ranging from 1000 to 30 000, comprising a) a polymer skeleton containing hydrocarbon-based repeating units provided with at least one heteroatom, and b) optionally functionalized pendent and/or terminal fatty chains containing from 12 to 120 carbon atoms and being linked to these hydrocarbon-based units, the aqueous phase, the liquid fatty phase and the polymer forming a physiologically acceptable medium.

2.-32. (canceled)
